(19) United States
(12) Patent Application Publication
(10) Pub. No.: US 2016/0238520 A1
(43) Pub. Date: Aug. 18, 2016

(54) DETERMINATION OF THE CONCENTRATION OF A COMPONENT IN ONE FLUID OF AN ANIMAL BY SPECTROSCOPIC ANALYSIS OF ANOTHER FLUID

(71) Applicant: BENTLEY INSTRUMENTS, Lille (FR)

(72) Inventor: Pierre BROUTIN, Lille (FR)

(21) Appl. No.: 15/029,163

(22) PCT Filed: Oct. 17, 2014
(86) PCT No.: PCT/FR2014/052650

§ 371 Date: Apr. 13, 2016

(30) Foreign Application Priority Data
Oct. 18, 2013 (FR) 1360145

(51) Int. Cl. G01N 21/27; G01N 33/487; G01N 21/3577

(52) U.S. Cl. CPC G01N 21/274; G01N 21/3577; G01N 33/487; G01N 2201/127

(57) ABSTRACT

Method for calibrating an infrared spectroscopy apparatus to produce spectra to determine the concentration of a component C1 in one fluid F1 of an animal, includes: a) obtaining samples of fluid F1 and of a different fluid from each animal belonging to a group of representative animals, fluid F2 containing detectable amounts of at least one component C2 which is directly or indirectly related to a metabolic pathway of component C1; b) measuring the concentration of component C1 in the samples of fluid F1; c) producing the complete IR absorption spectrum of the samples of fluid F2; d) identifying in the absorption spectra of fluid F2 samples the spectral ranges that correlate with the concentration of component C1 in fluid F1 samples; and e) calculating, on the basis of at least one of the correlating spectral ranges, a predictive mathematical model of the concentration of component C1 in fluid F1.

This invention relates to the field of methods of analysis of complex aqueous media and, more specifically, to that of the analysis of biological fluids by spectrometric techniques.

The subject matter of the invention concerns a method for calibrating an infrared spectroscopy apparatus to determine the concentration of a component C1 in one fluid F1 of an animal, in its blood for example, from spectroscopic data on components found in another fluid of the animal (F2), in its milk for example. The subject matter of the invention also concerns a routine method for determining the level of a compound C1 in the fluid F1 from a sample of the fluid F2.

The method advocated responds to a problem of broad scope, proposing to predict the contents of different components in a first fluid from the determination of components in another fluid. The latter can be easier to access than the first fluid, thus facilitating the provision of complete and reliable physiological data for the practitioner. This is the case, for example, with blood components, which are the indicators of numerous pathologies and whose concentration we wish to discover from the analysis of components of another fluid, milk or urine for example. The invention, which originated originally in the search for solutions in response to the high prevalence of cases of ketosis in dairy cows, soon proved to offer an approach of more general interest, applicable to various needs.

Acetonemia, also called ketosis, is a metabolic disease found mainly in high-producing dairy cows in early lactation. In both of its forms (clinical and subclinical), it gives rise to considerable economic losses in dairy farming, causing drops in production as well as fertility problems and an increase in postpartum diseases. In the case of subclinical ketosis in particular, dairy cows lose a lot of weight but there is no drop in production, as there is in the case of clinical ketosis. This acetonemia therefore passes unnoticed. An early and reliable diagnosis is therefore important.

In order to compensate for the energy deficit due to gestation and then lactation, the cow mobilizes its fat reserve in the form of Non-Esterified Fatty Acids (NEFA). These fatty acids are captured by the liver to be stored there in the form of triglycerides. Although the “normal” liver contains 1% triglycerides, 15 days before calving, the level of triglycerides can reach 5%. During the first month, depending on the energy deficit and in the absence of lipotropic factors, this fatty infiltration increases.

Now, triglycerides are not soluble in blood. They can only leave the liver and be transported in the blood in the form of Very Low Density Lipoproteins (VLDL). In principle, these lipoproteins are used chiefly for milk production but in the case of ketosis, and depending on the extent of mobilization (energy deficit) and insufficiency of lipotropic factors enabling the evacuation of triglycerides from the liver, a fatty accumulation builds up in the liver. If this is too great, the liver sheds the non-esterified fatty acids by oxidizing them and producing ketone bodies, namely acetone, acetoacetate and BHB (β-hydroxybutyrate), which end up in the blood at abnormal levels, and also, although to a lesser extent, in the milk. Subclinical acetonemia is considered to exist if the BHB level in the blood exceeds 1.4 mmol/l.

Various methods have been proposed, some of which are currently used to diagnose ketosis in dairy cows or to detect its prevalence in the herds. They are based on determining the components of the blood, milk or sometimes the urine of the animal, which undergo variations in the event of disease. The compounds sought as indicative of ketosis are ketone bodies, or rather, one of them in particular.

Indeed, although ketosis causes substantial changes in the concentration of the major and minor components of a dairy cow's blood and milk, and ketone bodies are minor, low-concentration, volatile and unstable parameters (chiefly acetone and acetoacetate), the determination of these bodies is the main route used to diagnose ketosis. The fat/protein content (F/PC) ratio is also commonly used as an indicator of acetonemia (F/PC>1.5) but this indicator is somewhat unreliable.

Several chemical methods aim to determine the concentration of ketone bodies in blood: gas chromatography, reaction kinetics associated with an absorption measurement, fluorimetry. These analyses, which are made on the basis of blood samples, require invasive sampling and the use of a laboratory, with delays and high costs.

A quantitative determination of ketone bodies from samples of milk is also possible. Chemical methods adopted in the laboratory (vanillin test for acetone, enzyme kinetics for acetone and BHB measured photometrically by the SKALAR method) also have their limitations: although they are reliable also in determining the minor components of milk, they are quite costly, slow and difficult to include routinely for a mass analysis. Moreover, these methods are restrictive as they only allow one specific molecule or a limited number of molecules, in this case ketone bodies, to be determined and take no account of the variations in concentration of the other components linked directly or indirectly to the metabolic problems at issue. There are some methods that can be used on the farm to test milk, blood or urine, but they are semi-quantitative methods (such as testing with colored strips).

Over recent years, IR spectroscopy methods have been developed. These are much faster and cheaper. They are commonly used to determine the concentration of milk compounds, which are the so-called major compounds, such as fats (F), proteins (P) and lactose; or so-called minor compounds, such as the fine fatty acid composition, protein fractions, urea and citric acid, but also ketone bodies.

We can cite, for example, the publication by Per Waaben Hansen “Screening of Dairy Cows for Ketosis by Uses of Infrared Spectroscopy and Multivariate Calibration” (J. Dairy Sci., 82:2005-2010), which describes a method of detecting ketosis by the determination of the concentration of acetone in the milk. Samples of milk are used to perform a calibration based on a correlation between, on the one hand, the milk's acetone concentration values obtained by a reference method and, on the other, the milk's acetone concentration values obtained by FTIR. The concentration of acetone in the milk is routinely measured with reference to these control samples.

However, using IR spectroscopy techniques to diagnose metabolic disorders on the basis of milk samples, especially in the case of ketosis, triggers a certain number of problems. In fact, the concentration of ketone bodies in milk is usually very low, even in the case of pathology. According to various studies, the tolerated limit of the BHB concentration is 0.07 mmol/l (i.e. 7.3 mg/l) in milk, whereas the maximum tolerated level of BHB in blood is 1.4 mmol/l (146 mg/l). In the study conducted for the present invention, the blood BHB level was on average 12.5 times higher than the milk BHB level.

In order to detect very low acetone and acetoacetate concentrations in fluids by infrared spectrometry, U.S. Pat. No. 6,385,549 proposes to perform a calibration of an FTIR spectrometer (spectrometer using Fourier Transform Infrared Techniques) from a batch of samples, some of which contain none or almost none of the compound for which the calibration is performed. The concentration of the compound sought, namely acetone, is below the theoretical detection limit of the apparatus. This method assumes that a large number of samples are available whose acetone and acetoacetate composition is known by reference methods. The calibration is developed to determine, only in the fluid analyzed, the concentration of the specific compound sought. An additional difficulty is that the high volatility of acetone and acetoacetate jeopardizes the reliability of the analysis, especially as these analyses are usually carried out on samples collected according to the milk recording procedures. The period between sampling and analysis can be very variable (from 1 to 6 days), as can the storage conditions (generally ranging from 4° C. to 40° C.), which affects the integrity of the samples. The results, and consequently the diagnosis, are significantly impaired.

Lastly, the reference method or direct method for detecting acetonemia in cows is still to measure the blood BHB level (this is the “gold standard”). Laboratory or portable dosimeters for measuring the blood BHB concentration work on the principle of Williamson's oxidation-reduction reaction, catalyzed by BHBDHase. The current generated at the electrode is proportional to the original amount of BHB. In the case of subclinical acetonemia, the threshold used is 1.4 mmol/l (i.e. 145.74 mg/l), with samples taken from a minimum of twelve cows. This method, which is the most commonly used, is invasive and requires the animal to be immobilized. It also requires the intervention of a qualified person such as a veterinary surgeon. Moreover, the daily variability of blood BHB can be very high, which means that several measurements would have to be taken in order to discover more accurately the development of the blood BHB of each animal.

The tests performed for the BHB determination, not in blood but in milk, have shown that the sensitivity of this dosimeter is too low because the milk BHB concentrations are often low. The correlation between the concentrations of blood BHB and milk BHB is low, especially in the highest concentrations, which are the marker of the disease. This correlation is also greatly affected by the periods between the two measurements (blood and milk) made within the milk recording, as well as by the method of storage. No satisfactory method is currently available.

There is therefore a need to offer farmers a systematic, non-invasive, simple and quick method, enabling the diagnosis, with a high degree of reliability, of ketosis and energy deficits in their animals, particularly in suckler cows and specifically in the lactation period of 3 to 7 weeks.

The present invention aims to meet this requirement by proposing a method whereby the diagnosis of acetonemia is made on the basis of quantitative measurements taken on the animal's milk. To do this, the level of BHB (or ketone bodies) in the blood is determined, using an infrared spectrum performed on a sample of milk. The proposed method involves searching for the existence of correlations between i) the characteristics of different ranges of the IR spectrum obtained for milk and ii) the variations in concentration of a compound sought in blood, without knowing the composition of the milk samples. These correlations are identified by multivariate analysis (PLS, PCR, ANN, MLR, etc.) and are incorporated into the prediction model. Thus all of the milk compounds that are directly or indirectly correlated to the desired metabolic variation sought are taken into account. Their direct or indirect link with the metabolism concerned is not necessarily known, the IR spectrum providing a footprint of the biological and physiological state of the animal when the milk was sampled.

It unexpectedly emerged that a quick and reliable prediction of the BHB level in blood could be achieved from an infrared spectrum performed on a milk sample, with sufficient accuracy for the potential and systematic detection of ketosis or energy deficit, even routinely when testing the milk, and with a high speed of analysis (up to 600 analyses/hour). This result was obtained due to a special method of calibrating the spectrometer and the development of a calibration model according to the invention. The recommended method has turned out to be of far-reaching interest since it allows the concentration of the different components of one body fluid to be predicted from the concentration determination of components in another fluid, such as milk or urine, thus providing the practitioner with comprehensive and reliable physiological data. This then enables the condition and changes in a herd or individual animals to be monitored, a better basis for diagnosis in the event of disease or metabolic disorder to be provided and preventive rather that curative measures to be adopted.

One aim of the invention is therefore to provide a method that can determine, from a sample of one fluid of an animal and from its spectrum, the level of a component (which may be a compound or a group of compounds) present in another fluid and which is an indicator of a normal or problematic condition affecting the animal. More specifically, one aim of the invention is to use samples of milk collected during conventional milk testing to determine the BHB level in an animal's blood, without having to take a blood sample. Note that the words “sample” and “fluid” describe different categories in order to clearly distinguish what pertains to the nature of the fluids and what pertains to a specific sample from a given fluid.

The invention therefore relates to a method for calibrating an infrared spectroscopy apparatus in order to produce spectra with a view to determining the concentration of a component C1 in a fluid F1 of an animal, which includes the following steps:
a) obtaining samples of said fluid F1 and of a fluid F2 other than the fluid F1 from each animal belonging to a group of representative animals, the fluid F2 containing detectable amounts of at least one component C2, which is directly or indirectly related to a metabolic pathway of the component C1,
b) measuring the concentration of the component C1 in the samples of fluid F1 by means of a reference method,
c) producing the complete IR absorption spectrum of the sample of fluid F2 in the frequency range from 650 cm⁻1 to 4000 cm⁻¹,
d) identifying in the absorption spectra of the samples of fluid F2, obtained in c), the spectral ranges that are correlated with the concentration of the component C1 in the samples of fluid F1, and
e) calculating, on the basis of at least one of said correlated spectral ranges, a predictive mathematical model of the concentration of the component C1 in the fluid F1.

The techniques based on infrared spectroscopy in the mid-infrared range (frequencies from 1000 cm⁻1 to 4000 cm⁻1) are already used in the agro-food industry and particularly in the dairy industry to determine the concentration of the major constituents in biological products such as milk, which are, for example, fats, proteins, lactose, but also those of minor components including urea, organic acids, free fatty acids, etc.

The total absorption of a sample is the sum of the absorptions of its individual components in each of their respective absorption ranges. However, if milk is taken as a specific example, due to its numerous components, the different peaks of the spectrum overlap. The amount of light absorbed in a given range is not specific to just one component, which makes it difficult, even impossible, to directly interpret the response of the spectrometer for each component and then prevents the accurate calculation of the concentration of a given component from the absorption of the sample.

In order to overcome this problem, a calibration is usually made that uses a mathematical model enabling the contribution of the different components to the absorption at a given frequency to be separated and their respective weight in the result of the measurement to be determined. In order to establish the calibration model, test samples representative of the medium are analyzed, on the one hand using a conventional chemical analysis and on the other with the aid of a spectrometric system, then the parameters of a mathematical relation between the two sets of results are defined. Once the calibration model is established, it is applied to the result of the spectrometric measurements to determine the concentration of a given component in a sample, starting from the amount of radiation absorbed at characteristic frequencies.

These methods are known per se so will not be described in further detail here. A person skilled in the art is capable of creating such models by the known means of multivariate calibration available. The applicable mathematical tools are, for example: Multiple Linear Regression or Multiple Nonlinear Regression (MLR or MnLR), Partial Least Squares Regression (PLS), Principal Component Regression (PCR) or Artificial Neural Network Regression (ANN). We now have such models for many compounds. The principle is that the spectra obtained for the fluid studied, and the concentration of a component of this fluid, are used to establish the predictive mathematical model. Once established, it enables us to determine the concentration of a component in any sample of said fluid, from just one infrared spectrum of this sample.

In an original way, according to the invention, spectra obtained for a fluid other than that containing the component sought serve as a basis for calculating the predictive model: it is from the absorption spectra of only the samples of the fluid F2 that a predictive model of the concentration of the component C1 in the fluid F1 is defined. Thus, at step e), at least one of the correlated spectral ranges is used as a basis to calculate a predictive mathematical model of the concentration of the component C1 in the fluid F1.

At step e), it is thus possible to select in the spectrum one or more, and preferably at least three correlated spectral ranges, and use said spectral ranges of the absorption spectra of the samples of the fluid F2 to calculate said predictive mathematical model of the concentration of the component C1 in the fluid F1. The correlated ranges selected preferably correspond to the spectral bands with the strongest correlation.

The samples of fluids F1 and F2 come from animals forming a representative group, in other words animals that constitute a collection reflecting the diversity of the herd in its environment (diversity as regards genetics, food, number of days of lactation, seasons, climates, etc.). The criteria for establishing a statistically representative group are known to a person skilled in the art. We would add that this group must also be representative as regards the metabolic state that is prompting the measurements (for example, when searching for a blood component that is a marker for lactation disorders, a group of animals made up of suckler cows will be chosen). The method and frequency of sampling must also be tailored to the problem to be diagnosed. This aspect proved important in order to obtain reliable predictive models. In the context of the present invention, it has been possible to show that there are significant variations in the level of blood BHB between the morning and evening, or depending on the animal's feed. This is made possible by the simplicity of sampling, which can be done when testing the milk, whether in the morning or the evening. The method used revealed and took these variations into account in order to obtain a robust and reliable calibration from the samples taken and from composite samples (mixture from the evening and morning milking, which is the sample normally used when testing the milk).

The fluid F2 must contain detectable amounts of at least one component C2 which has a direct or indirect relationship with a metabolic pathway of the component C1. A C2 component may be a product or a direct or indirect coproduct of the metabolism of the component C1, namely a product or coproduct of a reaction or chain of reactions where the component C1 is involved. In certain cases, the component C2 may be identical to the component C1. In general, the component C1 is connected with a particular metabolic, possibly pathological state to be diagnosed, of which it may be the cause or, by contrast, a consequence. This problem may also be of endogenous origin (genetic, disease, cow in gestation, energy deficit, number of lactations, etc.) or exogenous origin (feed, season, light conditions, etc.).

C2 components may be known as being products derived from the metabolic pathway of the component C1 and capable of ending up in the fluid F2, in amounts detectable by infrared spectroscopy. They may also not have been identified as such. The fact that there is a correlation between the components C2 of a fluid F2 and the concentration of a component C1 of a fluid F1 is found on completing the calibration method reveals the existence of a physiological effect, even though we do not always know what the mechanism for it is. Moreover, the inventive method may lead to the discovery of the existence of a physiological link between components not known until now.

One of the advantages of the method according to the invention is that it is not necessary to know beforehand the nature of the component or components C2 in order to establish the predictive model. Neither is it compulsory to identify them during the implementation of the method according to the invention although, as will be explained later, it is possible to do so and even particularly advantageous in order to increase the predictive value of the model and also discover new physiological relations.

The component C1 is chosen from those that are related to a particular physiological state of the animal, whether normal or pathological. Its presence in greater or smaller amounts in the fluid F1 is an indication of the state of the animal and measuring its concentration is a means of monitoring animals and/or diagnosing a deficiency, pathological disorder, etc. The reference method used for measuring its concentration at step c) is a commonly used method widely recognized by specialists as being the most accurate and reliable. It may be covered by a standard.

We have seen that, at step d), we identify on the absorption spectra of the samples of the fluid F2 obtained in step c), the spectral ranges that are correlated with the concentration of the component C1 in the samples of fluid F1. The correlated spectral ranges can be identified by calculating the correlation coefficient at each wavelength of the spectrum between the absorption value and the reference value of the component C1. According to a preferred characteristic of the invention, these ranges are identified by means of multivariate regression algorithms, in the interval of frequencies ranging from 650 cm⁻¹ to 4000 cm⁻¹. Preferably, said correlated spectral ranges are chosen in the frequency intervals ranging from 950 cm⁻¹ to 1620 cm⁻¹ and from 1680 cm⁻¹ to 3100 cm⁻¹, since we know that the area between 1620 cm⁻¹ and 1680 cm⁻¹ corresponds to one of the water absorption bands which is difficult to exploit.

According to a preferred characteristic of the method according to the invention, at step a), the samples of the fluids F1 and F2 obtained in step a) come from samples taken simultaneously from each animal in a group of representative animals, in other words they are taken from each animal in the group in a short period of time (for example within the same hour) and in any case in the same period of the circadian cycle. We recommend keeping the samples in cold storage and adding a preservative so that the fluid will not be denatured before the analyses can be conducted. In this way, the link that may exist between the compositions of the two fluids at a time t may be revealed by comparing the two samples, and this link is preserved even if the analyses are not performed straight away.

According to another preferred characteristic of the invention, steps b) to e) are performed using the undiluted samples of the fluids F1 and F2 taken in step a). We in fact recommended using natural samples of the fluid F2 in order to preserve the quantitative relationship between the components of each of the fluids F2 and F1. For example, natural milk samples will be used in order to maintain the link between blood composition and milk composition. Adulteration of the milk with BHB or other direct or indirect products or coproducts of the metabolism of the component C1 to create an artificial variation is not recommended as it would weaken the causal link.

As previously mentioned, the above-described method can be implemented without knowing the precise nature of the components of F2 for which a correlation is found in step d). However, and it is a considerable advantage of the present method, the infrared spectra obtained in step c) can be used in order to identify the components C2 specific to the fluid F2 whose concentration is linked to the concentration of the component C1 in the fluid F1.

It may be convenient, in order to identify the nature of the component or components C2, to link the level of correlation obtained with each spectral frequency. When the correlated spectral ranges are identified in step d), we calculate the coefficient of correlation (r) between each point of the spectra of the samples of the fluid F2 and the reference values of the concentration of the component C1 measured in the fluid F1. When we represent the variation of the correlation coefficients based on the frequency we obtain a graph, called the “correlation spectrum,” which shows the maximums at certain frequencies: these are the frequencies for which the correlation is strongest. If we compare the correlation spectrum with the infrared spectra of different compounds referred to in the data bases, we see that the peaks of some compounds overlap (absorption maximum) with the maximum correlation ranges: the compound in question heavily absorbs at the frequencies of the ranges strongly correlated with the component C1. This indicates, on the one hand, that this particular compound makes an important contribution to the calibration model and, on the other, that there is a relationship between the component C1 and this particular compound C2. This is how we have been able to show a significant correlation between the fatty acid profile of milk and certain fatty acids with the blood BHB.

Some components of the fluid F2 are known as being products obtained from the component C1 during metabolic reactions. For example, it is known that in the case of acetonemia, the blood BHB level increases and BHB is found in the milk, as well as fatty acids and other ketone bodies. Attention must be focused on these known components as being products of the metabolism of BHB (component C1) or associated directly or indirectly with the metabolic problems that cause variations in the BHB concentration. We can in particular produce a correlation spectrum and compare it with the infrared spectra of the various fatty acids, to reveal the instances of correspondence of maximums.

We can also look for a component C2, for which we do not know whether it has a metabolic relationship with the component C1, but for which we see that the absorption spectrum has one or more characteristic bands that coincide with one or more of the bands identified as being correlated with the component C1. We will now focus on the morphology of spectra (referred to in spectral data bases for example) to select the compounds C2 and determine a law of correlation between their concentration in the fluid F2 and the component C1 of the fluid F1.

Thus, according to the invention, the calibration method includes the following additional steps:
f) searching, among the components likely to be present in the fluid F2, for at least one component C2 having at least one characteristic absorption frequency that coincides with one of the bands located on the spectrum for which a correlation exists,
g) determining the concentration of said at least one component C2 in each of the samples of fluid F2,
h) calculating the correlation relation R1 existing between i) the concentrations of said at least one component C2 in the fluid F2 and ii) the concentrations C1 in the fluid F1.

This provides a second predictive mathematical model of the concentration of component C1 in the fluid F1, solely from the spectra of the fluid F2. It will allow the predictive power of the calibration method to be enhanced. The components likely to be present in the fluid F2 are those that a person skilled in the art recognizes as forming part of the composition of the fluid F2 of healthy or diseased animals. In a particularly original and advantageous way, step f) will also enable identification of the nature of the components C2 that had not until then been candidates to enter into the metabolic chain of the component C1.

Note that, alternatively, step f) can be omitted. It is possible to determine the nature of the component or components C2 correlated with the level of component C1 by directly calculating the coefficient of correlation between the values of C2 in the fluid F2 obtained with all of the calibrations already available and the compound C1. This step allows step f) to be skipped by searching for and identifying, among the components for which a calibration already exists (for example, complete fatty acid profile, organic acids, protein fractions, glycoproteins, carbohydrates, etc.) the nature of the compound C2 for which a correlation has been found.

According to a particular embodiment of the method according to the invention, at step g), the concentration of the said at least one component C2 in the samples of the fluid F2 can be determined from infrared spectra obtained at step c), with the aid of a predetermined mathematical model applying specifically to the said component C2. This is a step known to a person skilled in the art, who knows how to perform a multivariate calibration on the basis of a series of infrared spectra.

Note that, in order to be useable, the concentration of the component C2 in the samples of the fluid F2 must be determined during a routine regime, at the same time as the concentration of the other components. The infrared analyzer must therefore have a calibration specific to the component C2 that will allow the value of the concentration of C2 to be obtained by analysis of the infrared spectrum of the fluid F2. If the nature of C2 is known, pre-existing predictive models can be used. If this calibration is not already available, we will take the precaution of measuring the level of the component C2 in the fluid F2 by adopting the reference method for this component, and developing the corresponding model.

The above-descried step can be applied to a component such as a specific fatty acid, or to a group of components such as saturated or unsaturated fatty acids taken together. It can also take into account the combined contributions of several components through a complex indicator. In this context, steps f), g) and h) of the method according to the invention can be performed as follows:
f′) seeking among the components likely to be present in the fluid F2, n components C2n, each having at least one characteristic absorption frequency that coincides with at least one of the bands located in the spectrum for which a correlation exists,
g′) determining the concentration of said n components C2n in each of the samples of the fluid F2,
h′) calculating the value of an indicator lc proportional to the concentration of at least said n components C2n,
h″) calculating the correlation relation R2 existing between i) the values of the indicator lc in the fluid F2 and ii) the concentrations of the component C1 in the fluid F1.

The definition of such an indicator enables us to take into account different compounds found in the fluid F2 and produced via a metabolic pathway, always complex, it being possible for only one compound to be subject to variations or various influences. This is the case chiefly when a pathology exists. The marker compounds of this pathology can be found at very different concentrations in the fluids F1 and F2 of healthy animals and diseased animals.

Indicator lc is proportional to the concentration of at least the said n components C2n. It can thus be directly or inversely proportional to the level of a specific component C2. It can be a ratio of the levels of two components C2a and C2b for which a correlation exists, or the indicator lc can be based on one component C2n combined with another component not correlated with the component C1. Such an indicator can for example be the ratio between fats and protein content (F/PC), or even the ratio between certain fatty acids and the protein content, etc.

According to a preferred characteristic of the invention, the concentration of the said components C2n in the samples of the fluid F2 is determined on the basis of the infrared spectra obtained at step c), with the aid of a predetermined mathematical model applying to said components C2n. As stated above, the infrared analyzer must be equipped with a prediction model for each of the components C2n involved in the indicator, pre-existing or, if not available, developed on this occasion by a multivariate calibration method. If necessary, it must use the reference method to measure the concentration of each component C2n and develop the corresponding models.

According to an interesting characteristic of the method according to the invention, the fluid F1 is the blood of a healthy or diseased animal, and the fluid F2 is another fluid of the said healthy or diseased animal, either milk or urine. The group of animals chosen will be comprised according to best clinical practice, so as to have a representative sampling, to achieve a robust calibration. For example, to detect ketosis, cows from several farms in the region concerned will be selected and samples will be taken from the morning and evening milk from all of the cows between 3 and 7 weeks of lactation, several times a week for one month. The sampling must also be representative of the different breeds of animals and may also take into account the feed systems, which will be tested later by the model.

In the method according to the invention, the component C1 is preferably a substance whose concentration in the fluid F1 is an indication (direct or indirect) of a disorder or particular metabolic state. In particular, the component C1 can be a ketone body, a fatty acid, a carbohydrate, a protein, a glycoprotein or a hormone. It is known that ketone bodies are markers of acetonemia. Blood glucose is also often sought, as well as the level of hormones, particularly progesterone. Blood glycoproteins such as lactoferrin or Pregnancy-associated glycoproteins (PAG) are also sought. These analyses are currently conducted on blood samples but could be simplified or improved by indirect determination of derived compounds in the milk, thanks to the calibration method according to the invention.

Preferably, according to the invention, the component C1 is BHB (β-hydroxybutyrate), present in the blood of a healthy animal, in a state of energy deficit or in a state of acetonemia. In fact, BHB is much more stable than the other ketone bodies and for this reason is of greater interest: it has become the main marker of ketosis. However, as stated above, the measurement of its concentration in milk is unreliable because its level in milk is much lower than in blood (close or below the detection limit by mid-infrared spectroscopy). Furthermore, the BHB and acetone levels of milk fall very rapidly in the absence of refrigeration, a condition that cannot be easily fulfilled in the context of milk testing.

According to an interesting embodiment of the invention, said at least one component C2 can also be chosen from components known as having a direct or indirect relation with the metabolic pathway of the component C1. They can, for example, be known as being (direct or indirect) products of the metabolism of the component C1, or (directly or indirectly) associated with the metabolic problems of which we want to determine the concentration in the fluid F1. Some components of the fluid F2 are known as being products obtained during metabolic reactions from the component C1. The metabolism concerned can be normal or pathological, in which case the compound C2 is a marker of the metabolic problem sought. C2 is therefore in this case a product of a reaction or a chain of reactions of which the component C1 (or the metabolic problem) is the origin. Studies on animal metabolism provide indications concerning the physiological relations likely to provide these relations. However, until now, this knowledge has not been exploited for the indirect content determination of the compounds of blood (or another fluid). The compound or compounds C2 selected are analyzed on the basis of the same spectrum, using at least one absorption band of the component C2 concerned present in detectable quantities in the fluid F2. It goes without saying that an amount that is detectable in the fluid F2 may not be so in the fluid F1 (or detectable to an insufficiently reliable and accurate extent).

In an alternative or complementary manner, according to the invention, said at least one component C2 can be a newly identified component identified on completion of step f) as having a direct or indirect relation with a metabolic pathway of the component C1. A notable advantage of the present method is that it enables components whose relationship with a metabolic problem until then unknown to be identified, and its precise nature to be determined, thanks to the infrared spectra obtained in step c).

According to a particular embodiment of the method according to the invention, said at least one component C2 is a component present in the milk of a healthy animal, in a state of energy deficit or acetonemia, chosen from fats, proteins, glycoproteins and carbohydrates. The component C2 can in particular be a family of fatty acids or a specific fatty acid; the proteins, a family of proteins or a specific protein; the glycoproteins, a family of glycoproteins or a specific glycoprotein; the carbohydrates, a family of carbohydrates or a specific carbohydrate. The characteristic bands in the infrared spectra of these compounds or groups of compounds are documented, so that a person skilled in the art knows how to exploit the spectral data relating to them. For example, in the case of determining blood BHB, the component C2 can be a ketone body, a fatty acid or a family of fatty acids, since these compounds have a relationship with the metabolic pathway of BHB.

Preferably, according to the invention, tie said at least one component C2 is an unsaturated non-esterified fatty acid, preferably oleic acid or stearic acid. In fact, in the case of determining blood BHB from the spectrum of milk, we have established that there is an important correlation between the blood BHB level and the fatty acid profile of the milk (saturated fatty acids, unsaturated, mono- and polyunsaturated fatty acids. etc.) and particularly with unsaturated fatty acids and oleic and stearic acids.

In a particularly advantageous embodiment of the invention, the indicator lc is defined as being the ratio between the level of a fatty acid or a class of fatty acids (saturated or unsaturated fatty acids for example) and the protein content.

The method described above is designed to calibrate an infrared spectroscopy apparatus in order to perform routine analyses in farms or analyses in the context of scientific studies. This is why another aim of the present invention is a method for determining the concentration of a compound C1 in a fluid F1 of an animal, using an infrared spectroscopy apparatus to produce absorption spectra, a method that involves using a method of calibrating said spectroscopy apparatus as described below, in order to record (all) the spectral information of a sample of a fluid F2 of said animal, other than the fluid F1, said fluid F2 containing at least one component C2 having a direct or indirect relationship with a metabolic pathway of the component C1.

As explained above, the component C1 may be at the origin of the metabolic pathway leading to C2, or it may result from a particular metabolic problem that is sought after. The concentration of C1 in the fluid F1 is directly determined, using a spectrum produced with the sample of fluid F2, without having identified the nature of the component C2. Several C2 components, produced directly or indirectly from the metabolism of the component C1, may be present in the fluid F2 and may each serve, separately or together, to predict the level of C1 in the fluid F1, since all of the components present in the fluid F2 leave their fingerprint on the infrared spectrum.

The concentration of a component C1 in a fluid F1 of an animal can also be determined from components C2 of the fluid F2, whose nature has been identified previously. According to this variation of the invention, the method of determining the concentration of a component C1 in a fluid F1 of an animal, using an infrared spectroscopy apparatus to produce absorption spectra, includes the use of a calibration method of said spectroscopy apparatus as described above to measure in a sample of a fluid F2 of said animal, other than the fluid F1, detectable amounts of at least one specific component C2, which is a direct or indirect product of the metabolism of the component C1. The component C2 is here predetermined in its nature, in other words it is already known to the prior art or has previously been identified when implementing the present method of determination. This knowledge of the nature of the component C2 will influence the choice of the relevant spectral ranges used for calibration prior to routine determinations, and will enhance their predictive power.

A more specific aim of the present invention is a method for determining the concentration of BHB in the blood of an animal, using an infrared spectroscopy method to produce absorption spectra, the method including the use of a method of calibrating an infrared spectroscopy apparatus to measure in a sample of milk of said animal a detectable concentration of at least one component, chosen from unsaturated fatty acids, preferably oleic acid or stearic acid, and the protein content. The blood BHB concentration is thus determined directly from the spectrum of the milk.

Thanks to the method described here, it is now easy to systematically monitor the state of health of the animals or their physiological state in the context of preventing disorders such as acetonemia in sucker cows. Indirect determination of the blood BHB level may be used to monitor the development of the blood BHB level of an animal over time (from one test to the next) and also to compare the BHB level of one animal against the BHB levels of the herd. An animal in a state of acetonemia is thus quickly identified.

The method according to the invention has numerous applications. For example, it can be applied when determining the glycoprotein concentration (PAG) of the blood from a spectroscopic analysis of a sample of an animal's blood in order to determine quickly, at an early stage and accurately the PAG concentration of the blood and thus its state of pregnancy.

Thanks to the method according to the invention, four approaches are now possible:

1. Directly determining the component C1 of F1 from the spectrum of F2 (milk) on the basis of a multivariate calibration.
2. Identifying C2 components correlated directly or indirectly to component C1 in order to predict C1 directly or in relation to 1) so as to improve diagnosis of the metabolic problem.
3. Using a ratio of C2 components (indicator lc) correlated directly or indirectly to the component C1 in order to predict C1 directly or in relation to 1) so as to improve diagnosis of the metabolic problem.
4. Directly determining the component C1 in F1 in relation to (2) or (3).

Generally, the method according to the present invention is much faster and cheaper than the methods previously used. It enables non-invasive analyses that can be systematized and frequently repeated. It enables a simultaneous analysis of all of the components of the medium subjected to spectroscopy, unlike conventional chemical methods. It opens the possibility of devising a complete diagnosis (fat, proteins, lactose, fatty acid profile of the milk, etc.).

Further features and advantages of the invention will emerge from the following description of an embodiment shown in the accompanying drawings, in which:

FIG. 1 represents the relationship between the values of the blood BHB concentration obtained by direct chemical measurement (reference method), and the values of the blood BHB obtained by a predictive model obtained from the infrared spectrum of 696 samples of milk, according to the invention.

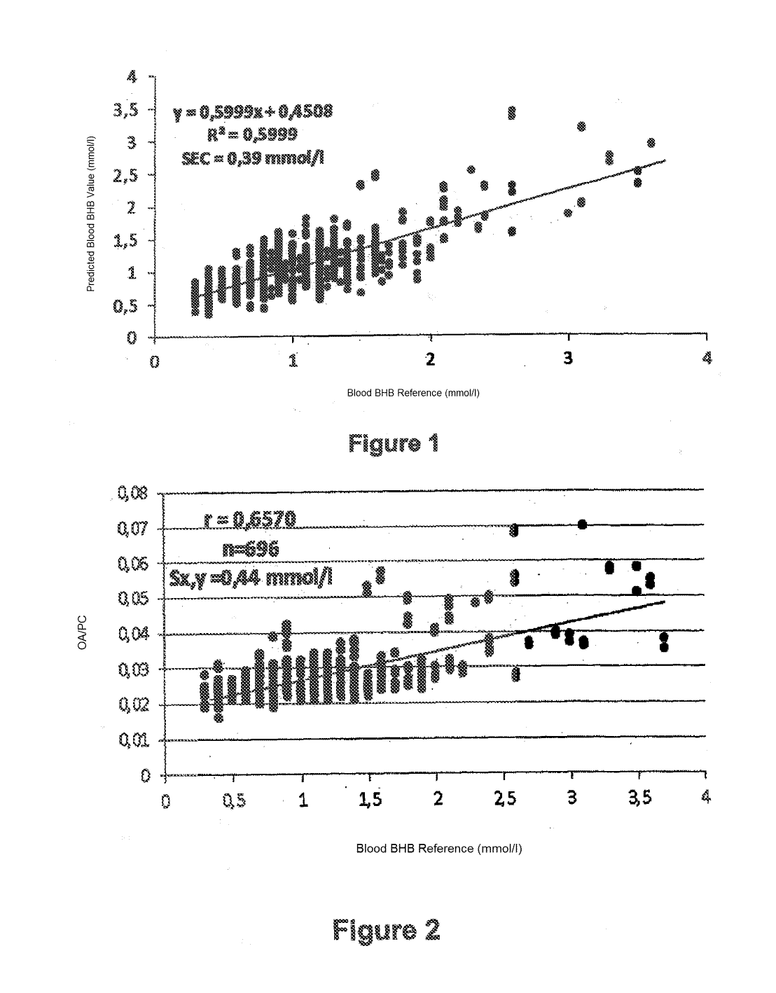

FIG. 2 represents the development of the Oleic Acid (OA)/Protein Content (PC) indicator for the prediction of ketosis from the IR spectrum of 696 samples of milk.

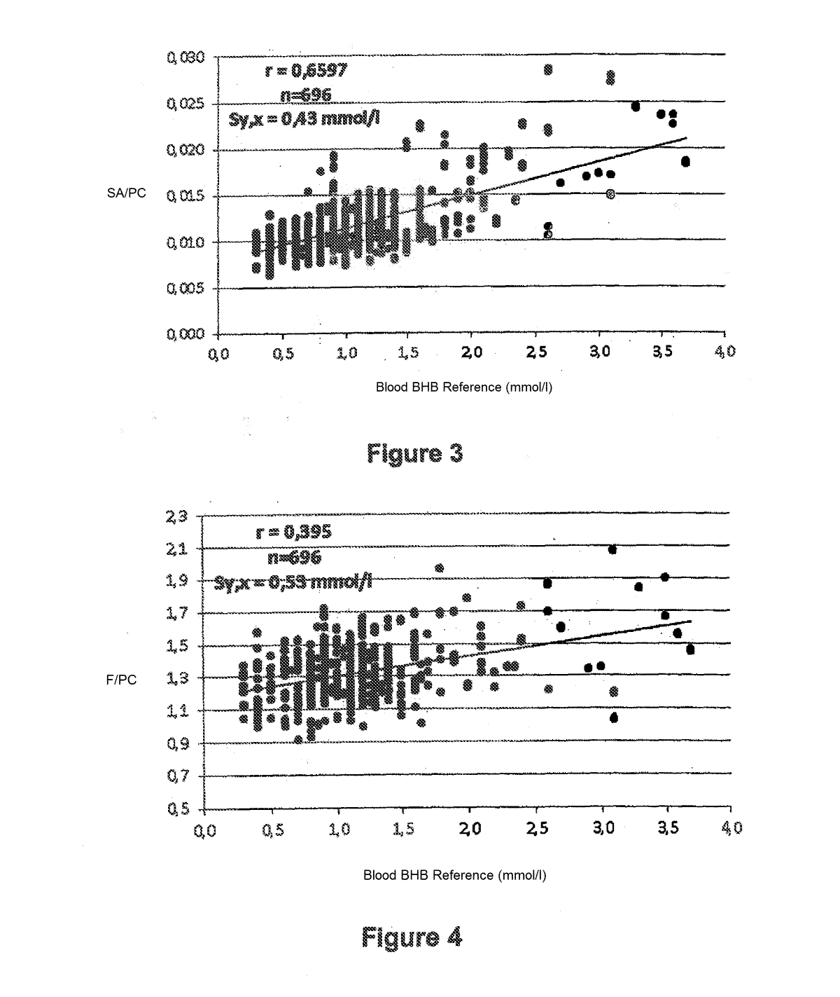

FIG. 3 represents the development of the Stearic Acid (SA)/Protein Content (PC) indicator for the prediction of ketosis from the IR spectrum of 696 samples of milk.

FIG. 4 represents the use of the F/PC ratio for the prediction of ketosis from the IR spectrum of 696 samples of milk, (unsatisfactory).

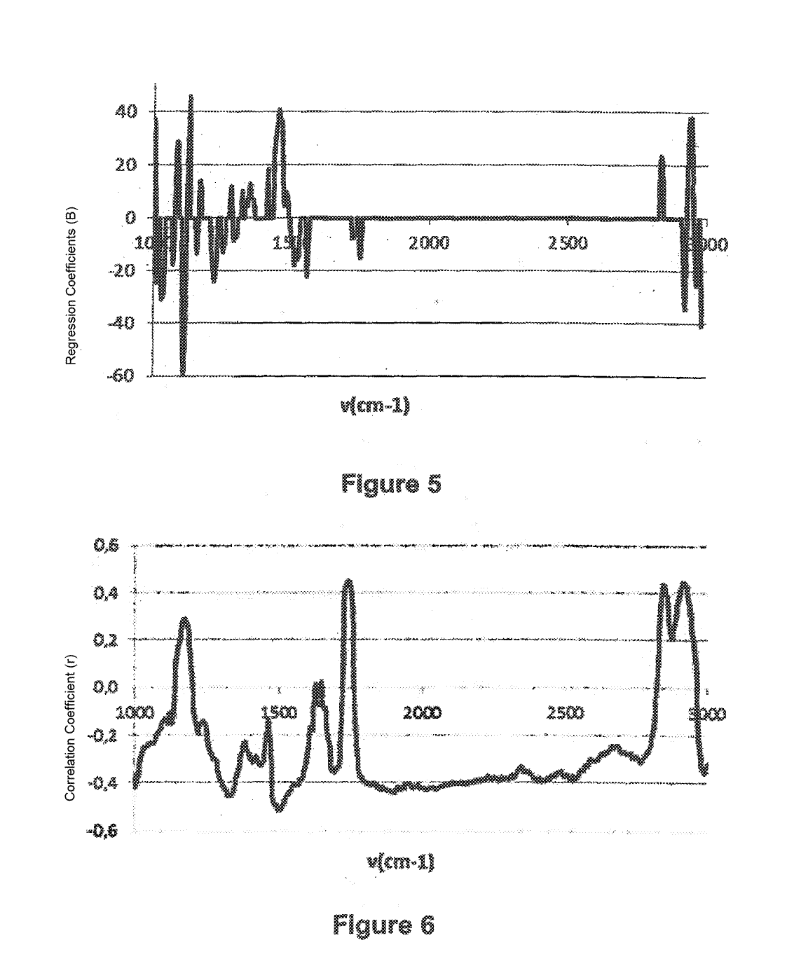

FIG. 5 represents the B regression coefficients of the model, based on the absorption frequency.

FIG. 6 represents the correlation spectrum showing the variation between i) the correlation coefficients between blood BHB and the infrared spectrum of milk.

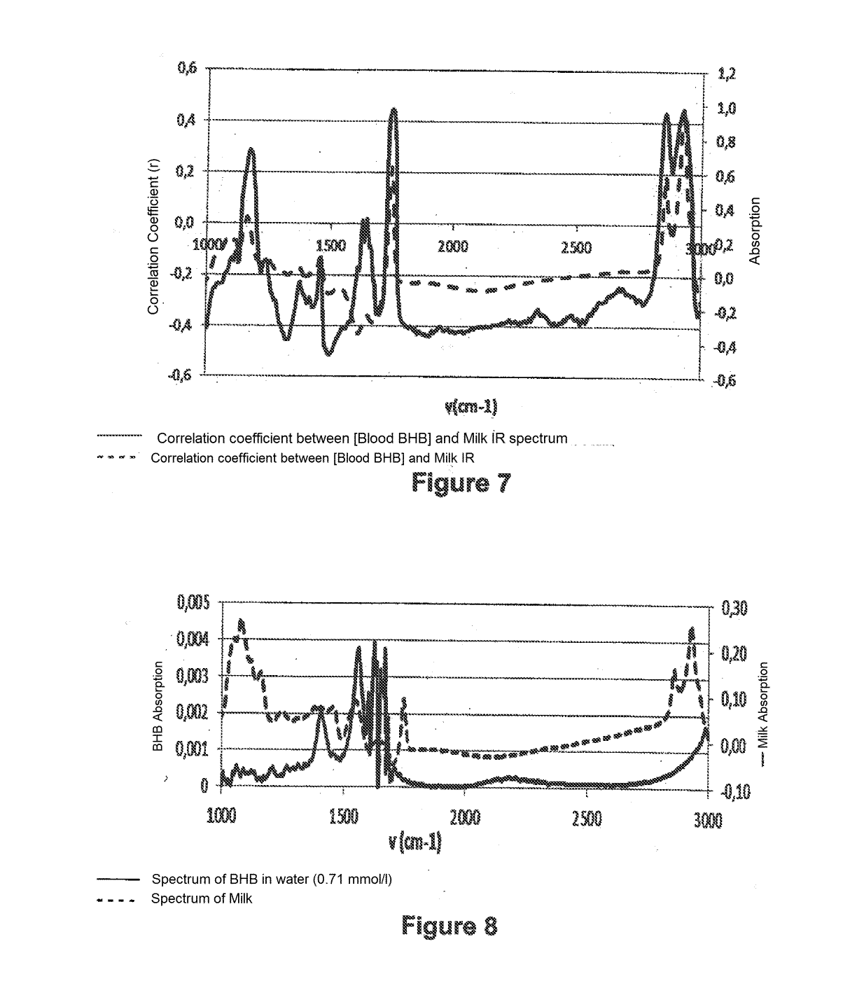

FIG. 7 shows the overlap of the same correlation spectrum on the infrared spectrum of a sample of cream.

FIG. 8 is an infrared absorption spectrum of BHB in water at the concentration of 0.71 mmol/l (73.9 mg/l) and a spectrum of milk.

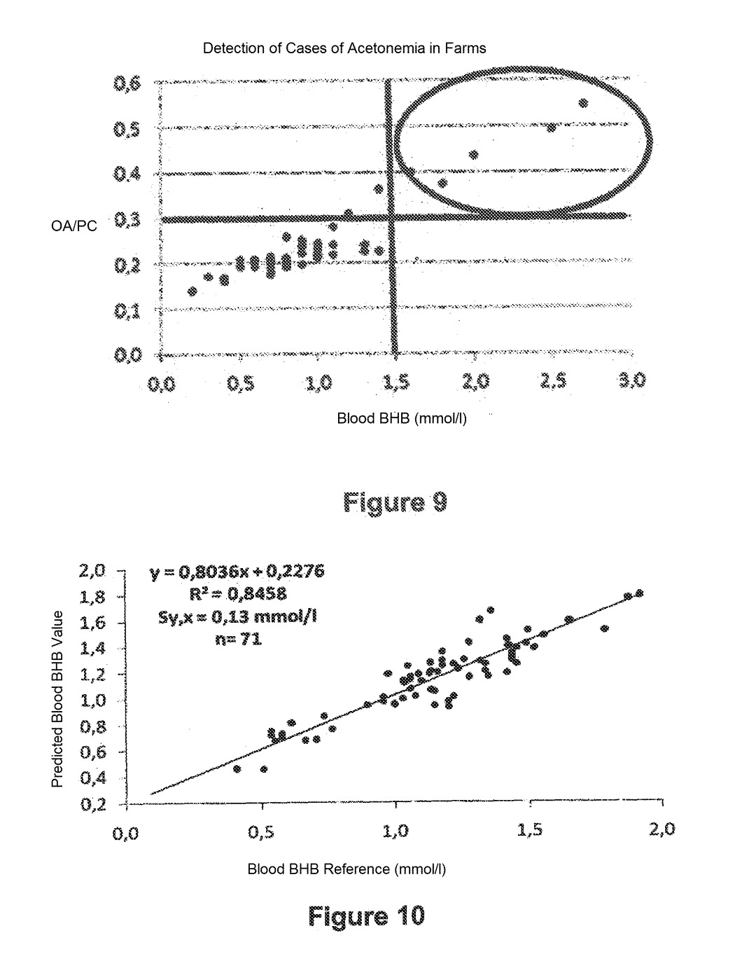

FIG. 9 represents a two-dimensional map of the detection thresholds for the two variables OA and PC.

FIG. 10 is a graph showing the improvement of the prediction of blood BHB on the scale of the herd.

### EXAMPLE 1

The procedure described below was carried out to calibrate a spectrometer, in order to determine the BHB level in the blood of suckler cows, from infrared spectra of milk samples.

1) At least 100 milk samples are collected, preferably from 100 to 300 samples, each corresponding to an individual cow. Sampling is done taking into account the various feed systems, various breeds and the metabolic problem to be detected. For example, by selecting healthy cows and those in a state of acetonemia. Ideally, 300 to 1000 representative samples are collected from the collection area.
2) The milk samples are refrigerated at 4° C. immediately after collection in order to maintain their integrity and prevent the evaporation of certain components. In fact, the concentration of ketone bodies in milk decreases very rapidly, in a few hours, in the absence of refrigeration. A preservative is added to the samples in order to maintain their integrity until they are analyzed.
3) At the same time as the milk, a sample of blood is taken from each cow, in order to maintain the time/causal/metabolic link between the composition of the milk and blood and to obtain an optimal correlation. It is refrigerated immediately.
4) Analyses are conducted on the BHB levels in the blood samples according to the reference method, in this case by using the OPTIUM XCEED® meter. The values obtained are stored in a memory of a central unit.
5) The full spectrum (650-4000 cm⁻¹) of the milk samples is produced on a mid-infrared Fourier Transform (FTIR) analyzer. The samples are first heated in a water-bath to a temperature of between 38° C. and 42° C. for a maximum of 20 minutes in order to homogenize the fat in the bottle. The infrared analyzer is equipped with a high-pressure pump and a homogenizer in order to break down the fat globules, ensuring that their diameter is smaller than the shortest wavelength used in developing the calibration model. These experimental conditions are important because the spectral range (2800-3000 cm⁻¹) used to determine the fatty acid profile of the milk is very sensitive to the size of fat globules. The infrared spectra are automatically recorded in the central unit.
6) The BHB values obtained by the reference method are edited in the chemometric software and compared with the spectra in order to develop the calibration model: to define the spectral areas and the regression B coefficients of the models enabling the blood BHB concentration of the animal to be predicted on the basis of its milk sample.

FIG. 5 represents the B coefficients of the model. FIG. 1 shows the predictive value of the model thus developed, by comparing the values of the BHB concentrations measured in the blood with those obtained from the infrared spectra of the milk.

We would point out that the calibration model was developed on the basis of natural milk samples, representative of animals suffering from the metabolic disorders that we were trying to diagnose. A prior selection was made after a detailed study of representative cows from several farms in the region (<100 days' lactation). Samples of milk were taken as part of the milk test (morning and/or evening) from all of the cows selected, several times a week for one month (656 samples taken), completely filling the analysis bottles (no dead volume so as to limit evaporation). The blood samples were taken near the tail during each milk sampling. The milk samples were immediately refrigerated at 4° C. and a preservative such as Bronopol (2-bromo-2-nitropropane-1,3-diol) was added in order to limit evaporation of the ketone bodies.

### Results

It emerged that a quick and reliable prediction of the blood BHB level from an infrared spectrum of a milk sample was possible. The above method was adopted on 696 samples of cow's milk. The blood BHB calibration model developed (see FIG. 1) from infrared spectra of milk and the blood BHB concentration gives a correlation coefficient (r) of 0.7370 and a prediction standard deviation of 0.39 mmol/l (40.6 mg/I). This accuracy is sufficient for the potential and systematic routine detection of ketosis during milk testing at a maximum speed of analysis of 600 analyses/hour.

Our work has shown that the daily variation (morning/evening) of the blood BHB level could be very great. The choice of sampling has played a role in the success of the development of a robust calibration model that incorporates not only the variations in concentration of the ketone bodies but also the variations in concentration of the other major or minor components resulting from the targeted metabolic disorder, in particular the variations in the fatty acid composition. The mid-infrared spectrum is a perfect fingerprint of the chemical and organic composition of milk and a mirror of the general state of the cow. It is therefore very important to develop calibration models based on samples of natural milk, representative of healthy cows and cows suffering from these metabolic disorders.

### EXAMPLE 2

An identification of the C2 components of the milk for which a correlation exists with the blood BHB concentration has been made. On the basis of the spectra of milk samples, the spectral ranges that are correlated with the blood BHB concentration have been determined (and they have been incorporated into the prediction model, as explained in Example 1).

In order to determine the nature of the C2 components of milk (or of an indicator lc) correlated to the BHB level, the following procedure was adopted:
Calculation of the correlation coefficient between each point of the milk spectra and the BHB reference values.
Graphic representation of the “correlation spectrum” (see FIG. 6): this shows which spectral ranges are correlated to the blood BHB.
Comparison with the infrared spectrum of the different components of milk. The spectrum of a sample of cream was compared with the correlation spectrum (see FIG. 7).

The “correlation spectrum” obtained clearly shows the spectral ranges associated with the fat and consequently the fatty acid profile of the milk. The best-correlated spectral bands between the spectra of milk and the reference values of blood BHB correspond to the main absorption bands of the fatty acids, namely 1170 cm⁻¹, 1380 cm⁻¹, 1450 cm⁻¹, 1750 cm⁻¹, 2850 cm⁻¹ and 2930 cm⁻¹. The correlation spectrum therefore shows that the fatty acid profile of the milk has a direct or indirect preponderant link (explanatory variable) with the blood BHB level.

This result could not be obtained directly from the BHB concentration in the milk. In fact, as shown in FIG. 9, the BHB spectrum has two characteristic absorption bands at 1400 cm⁻¹ and 1560 cm⁻¹. The correlation spectrum between the milk and blood BHB spectra (FIG. 8) reveals no particular correspondence at the characteristic peaks of BHB. The blood BHB level is much more closely correlated to the fatty acid composition of the milk than to the BHB level of the milk.

### EXAMPLE 3

We then calculated the correlation coefficients of the various fatty acids present in the milk and the BHB concentration in the blood. This step allowed us to determine which fatty acids of the milk were most closely correlated to the BHB level of the blood and to use them as indicators to refine the diagnosis of ketosis or an energy deficit.

The fatty acids for which the correlation was strongest were oleic acid and stearic acid. Although the presence of oleic acid and its correlation to the blood BHB level was expected for known physiological reasons, our study has highlighted the interest of other fatty acids, like stearic acid. Out of the 696 samples analyzed, the correlation coefficients between the oleic acid and stearic acid level with the blood BHB level are 0.52 and 0.58 respectively. We also see that these two fatty acids, and the general fatty acid profile of the milk, carry significant weight in the blood BHB prediction model. The method according to the invention has also enabled the most efficient lc indicators to be defined. It has proved that the OA/PC (oleic acid content/protein content) and SA/PC (stearic acid content/protein content) ratios were better indicators of ketosis than the F/PC (fat/protein content) ratio currently used. We would point out that an F/PC ratio of >1.5 is usually an indicator of ketosis. Out of the 696 samples analyzed, we have obtained a correlation (r) between OA/PC, SA/PC and the blood BHB levels measured by the Optium Xceed® meter of 0.66 (FIG. 2) and 0.66 (FIG. 3) respectively. By contrast, the correlation between F/PC and the blood BHB levels were only 0.39 (FIG. 4). We therefore observe an improvement of 40.9% compared to the F/PC indicator currently used.

The determination of certain milk components C2 correlated directly or indirectly to BHB (in the case of the lc indicator=OA/PC) has allowed us to further refine the diagnosis of acetonemia. We can in fact establish detection thresholds for both variables OA/PC and BHB, and create a two-dimensional map as shown in FIG. 9. The samples/animals corresponding to the circled dots in the graph are in a state of acetonemia. Farmers thus possess a tool to diagnose acetonemia.

### CONCLUSION

The spectral ranges correlated with the concentration of the compound sought have been directly identified by multivariate regression algorithms (such as PLS) with calculation of the regression B coefficients. The calculation of the correlation spectrum can be used instead of B coefficients, or even in addition to them, in order to try and determine the nature of the component C2 correlated to the component C1, in this case the nature of the fatty acid. The correlation spectrum has shown that it is the fatty acid profile of milk that was most closely correlated to the blood BHB level. The concentration of the main fatty acids of the milk was then determined by multivariate calibrations and the one/those that were the most closely correlated to the blood BHB were determined.

### EXAMPLE 4

We have seen that the detection of ketone bodies in milk is an unreliable method, the sole aim of which is to determine the concentration of this compound in the milk, and only taking into account this variable to try and explain the metabolic disorder. By contrast, the invention is based on an approach that takes into account all of the variables in order to establish a direct link between the composition of the milk and the concentration of BHB (component C1) in the blood. The predictive model includes the traces of ketone bodies that may be present in the milk, but more especially all of the variables and data that are directly or indirectly linked to blood BHB and to the metabolic problem to be diagnosed. Consequently, the prediction thereof is significantly improved.

For example, the improvement in the prediction of blood BHB on the scale of a herd or per group of cows has been proved by calculating the reference average and the predicted values per herd. The results are shown in the graph in FIG. 10.

The warning level for blood BHB is typically 1.4 mmol/l. Herds with an acetonemia problem or energy deficit can thus be easily detected thanks to our method.

1-20. (canceled)

21. A method for calibrating an infrared spectroscopy apparatus in order to produce spectra with a view to determining the concentration of a component C1 in a fluid F1 of an animal, said method comprising:
a) obtaining samples of said fluid F1 and of a fluid F2 other than the fluid F1 from each animal belonging to a group of representative animals, the fluid F2 containing detectable amounts of at least one component C2 which is directly or indirectly related to a metabolic pathway of the component C1,
b) measuring the concentration of the component C1 in the samples of fluid F1 by means of a reference method,
c) producing the complete IR absorption spectrum of the sample of fluid F2 in the frequency range from 650 cm⁻¹ to 4000 cm⁻¹,
d) identifying in the absorption spectra of the samples of fluid F2, obtained in c), the spectral ranges that are correlated with the concentration of the component C1 in the samples of fluid F1, and
e) calculating, on the basis of at least one of said correlated spectral ranges, a predictive mathematical model of the concentration of the component C1 in the fluid F1.

22. The method according to claim 21, wherein at step d), said correlated spectral ranges are identified by means of multivariate regression algorithms, in the interval of frequencies ranging from 650 cm⁻¹ to 4000 cm⁻¹.

23. The method according to claim 21, wherein the samples of the fluids F1 and F2 obtained in step a) come from samples taken simultaneously from each animal in a group of representative animals.

24. The method according to claim 21, wherein steps b) to e) are performed using the undiluted samples of the fluids F1 and F2.

25. The method according to claim 21, further comprising the following additional steps:
f) searching, among the components likely to be present in the fluid F2, for at least one component C2 having at least one characteristic absorption frequency that coincides with one of the bands located on the spectrum for which a correlation exists,
g) determining the concentration of said at least one component C2 in each of the samples of fluid F2,
h) calculating the correlation relation R1 existing between i) the concentrations of said at least one component C2 in the fluid F2 and ii) the concentrations C1 in the fluid F1.

26. The method according to claim 25, wherein the concentration of said at least one component C2 in the samples of the fluid F2 can be determined from infrared spectra obtained at step c), with the aid of a predetermined mathematical model applying specifically to said component C2.

27. The method according to claim 21, further comprising the following additional steps:
f′) seeking among the components likely to be present in the fluid F2, n components C2n, each having at least one characteristic absorption frequency that coincides with at least one of the bands located in the spectrum for which a correlation exists,
g′) determining the concentration of said n components C2n in each of the samples of the fluid F2,
h′) calculating the value of an indicator lc proportional to the concentration of at least one of said n components C2n,
h″) calculating the correlation relation R2 existing between i) the values of the indicator lc in the fluid F2 and ii) the concentrations of the component C1 in the fluid F1.

28. The method according to claim 27, wherein the concentration of said components C2n in the samples of the fluid F2 is determined on the basis of the infrared spectra obtained at step c), with the aid of a predetermined mathematical model applying to said components C2n.

29. The method according to claim 21, wherein the fluid F1 is the blood of a healthy or diseased animal, and the fluid F2 is another fluid of said healthy or diseased animal, either milk or urine.

30. The method according to claim 21, wherein the component C1 is a substance whose concentration in the fluid F1 is a direct or indirect indication of a disorder or particular metabolic state.

31. The method according to claim 30, wherein the component C1 is selected from among a ketone body, a fatty acid, a carbohydrate, a protein, a glycoprotein and a hormone.

32. The method according to claim 31, wherein the component C1 is BHB (β-hydroxybutyrate), present in the blood of a healthy animal, in a state of energy deficit or in a state of acetonemia.

33. The method according to claim 25, wherein the at least one component C2 is chosen from components known to have a direct or indirect relationship with a metabolic pathway of the component C1.

34. The method according to claim 25, wherein said at least one component C2 is a newly identified component identified on completion of step f) as having a direct or indirect relation with a metabolic pathway of the component C1.

35. The method according to claim 21, wherein said at least one component C2 is a component present in the milk of a healthy animal, in a state of energy deficit or acetonemia, selected from among from fatty acids, proteins, glycoproteins and carbohydrates.

36. The method according to claim 35, wherein said at least one component C2 is an unsaturated non-esterified fatty acid.

37. The method according to claim 29, wherein the indicator lc is defined as being the ratio between the level of a fatty acid or a class of fatty acids and the protein content.

38. A method for determining the concentration of a component C1 in a fluid F1 of an animal, using an infrared spectroscopy apparatus to produce absorption spectra, which comprises using a method of calibrating said spectroscopy apparatus according to claim 21, in order to record the spectral information of a sample of a fluid F2 of said animal, other than the fluid F1, said fluid F2 containing at least one component C2 having a direct or indirect relationship with a metabolic pathway of the component C1.

39. A method for determining the concentration of a component C1 in a fluid F1 of an animal, using an infrared spectroscopy apparatus to produce absorption spectra, which comprises using a method of calibrating said spectroscopy apparatus according to claim 21, in order to measure in a sample of a fluid F2 of said animal, other than the fluid F1, detectable amounts of at least one specific component C2, having a direct or indirect relationship with a metabolic pathway of the component C1.

40. The method according to claim 39, wherein the concentration of BHB in the blood of an animal is determined by measuring in a sample of milk of said animal a detectable concentration of at least one component, selected from among unsaturated fatty acids, and the protein content.
